(19) United States
(12) Patent Application Publication
(10) Pub. No.: US 2008/0044395 A1
(43) Pub. Date: Feb. 21, 2008

(54) In Vitro Method for Isolating, Proliferating and Differentiating Germ-Line Stem Cells

(75) Inventors: Kye-Seong Kim, Seoul (KR); Dong-Ryul Lee, Seoul (KR)

Correspondence Address: ROTHWELL, FIGG, ERNST & MANBECK, P.C., 1425 K STREET, N.W., SUITE 800, WASHINGTON, DC, 20005, US

(73) Assignees: SUNGKWANG EDUCATIONAL FOUNDATION, Pochon-siu (KR); CHABIOTECH CO., LTD., Seoul (KR)

(21) Appl. No.: 11/578,190

(22) PCT Filed: Apr. 13, 2004
(86) PCT No.: PCT/KR04/00850

§ 371 Date: Oct. 10, 2006

(51) Int. Cl. A61K 48/00; A61P 15/08; C12N 5/00; C12N 5/08

(52) U.S. Cl. 424/93.21; 435/366; 435/383

(57) ABSTRACT

The present invention discloses methods for isolating, proliferating and differentiating germ-line stem cell in vitro. More specifically, the present invention discloses a method for the in vitro isolation and proliferation of mammalian germ-line stem cells, characterized in culturing cells isolated from mammalian testis in an embryonic stem cell culture medium, and for the in vitro differentiation, characterized in encapsulating mammalian germ-line stem cells and Sertoli cells with calcium alginate and co-culturing with peritubular cells. Also, the present invention discloses germ-line stem cells obtained by above methods, a composition for the treatment of male infertility, comprising the germ-line stem cells, and a method for the treatment of male infertility by the use of the germ-line stem cells.

### TECHNICAL FIELD

The present invention relates to methods for isolating, proliferating and differentiating mammalian male germ-line stem cells (GSCS) in vitro.

### BACKGROUND ART

Male germ-line stem cells are a kind of adult stem cells and known to have unipotency devoted solely to the generation of sperm by self-renewal and differentiation. The germ-line stem cells are rare relatively quiescent population that lies in a protected region in the testis among supporting cells, and their proliferation and differentiation are regulated by communications with external signals.

The processes of development and differentiation of the germ-line stem cells are well known. For example, the primordial germ cells of mice appear in the epiblast at 7.5 dpc (days post coitum), proliferate and migrate to the genital ridge. Then, these cells form gonads at 12.5 dpc and differentiate into gonocytes. The gonocytes continue to proliferate until 16 dpc, and then stop to proliferate. After birth, the gonocytes are differentiated into undifferentiated type A spermatogonia. The spermatogonia can continue to produce sperm by self-renewal and differentiation after the puberty (Kierszenbaum et al., Endocr Rev 15:116-134, 1994).

Spermatogenesis can be divided into three processes: 1) spermatocytogenesis where spermatocytes are produced from spermatogonia by mitosis; 2) meiosis where spermatids are produced from the spermatocytes; and 3) spermiogenesis where sperm are produced from the spermatids. The spermatogenesis is regulated by the follicle stimulating hormone (FSH) and luteinizing hormone (LH) secreted from the pituitary body. LH act on Leydig cells, the interstitial cells of testis, so as to regulate the production of testosterone, the male hormone. FSH is involved in the spermatogenesis by regulating the function of Sertoli cells which physically and biochemically support germ cells.

Germ-line stem cells have been used in the study for searching the factors related with the self-renewal and differentiation of stem cells and their mechanism. At the present time where studies on human embryonic stem cells (Thomson et al., Science 282: 1145-1147, 1998) and human embryonic germ cells (Shamblott et al., Proc Natl Acad Sci USA 95: 13726-13731, 1998) have been progressed. Germ-line stem cells remain as a main research field for the analysis of the characteristics of stem cells. Furthermore, the possibility of the use of germ-line stem cells for the treatment of human male infertility due to failure in germ cell proliferation and differentiation and for the study on the production of transgenic animals with genetically modified animal sperm (Nagano et al., Biol Reprod 64: 1409-1416, 2001; Hamra et al., Proc Natl Acad Sci USA 99: 14931-14936, 2002).

Because of the above-described utility of germ-line stem cells, attempts to isolate, proliferate and differentiate the germ-line stem cells in vitro have been continued since the early 1960s. For example, there is a report that the production of round spermatids can be made by introducing TERT genes into the germ-line stem cells of mice to immortalize the cells and inducing the long-term proliferation and differentiation of the immortalized cells in vitro (Feng et al., Science 297: 392-395, 2002). However, the clinical use of the round spermatids produced from the immortalized cells has a potential risk. Also, it was found that germ-line stem cells can be differentiated into sperm by performing the long-term culture of germ-line stem cells in vitro and transplanting the cultured cells again into testis (Kanatsu-shinohara et al., Biol Reprod 69: 61-6, 2003).

However, despite of the above-described efforts, there is a difficulty in the isolation of germ-line stem cells since the amount of germ-line stem cells present in vivo is very small and the development of a specific marker for the isolation of germ-line stem cells is not yet made. Also, because of the difficulty in isolation, a technical system allowing the mass proliferation of germ-line stem cells by in vitro culture is not yet completed.

Accordingly, there is an urgent need for the development of isolation and proliferation method of a large amount of mammalian germ-line stem cells in vitro and an effective differentiation method of germ-line stem cells.

### DISCLOSURE OF THE INVENTION

During extensive studies on methods for isolating, proliferating and differentiating germ-line stem cells from mammalian testis, the present inventors have found that when cells isolated from mammalian testis are cultured in an embryonic stem cell culture medium, the germ-line stem cells will form a mass of cell colonies so as to effectively achieve the isolation and proliferation of the germ-line stem cells at the same time. The present inventors have also found that when the isolated germ-line stem cells are encapsulated with calcium alginate and co-cultured with peritubular cells, the differentiation of the germ-line stem cells is effectively achieved. On the basis of these findings, the present invention was completed.

Therefore, it is an object of the present invention to provide a method for effectively isolating and proliferating germ-line stem cells from mammalian testis.

Another object of the present invention is to provide a method for effectively differentiating germ-line stem cells in vitro.

Still another object of the present invention is to provide germ-line stem cells obtained by said methods.

Still another object of the present invention is to provide a composition for the treatment of male infertility.

Still another object of the present invention is to provide a method for the treatment of male infertility.

Still another object of the present invention is to provide a method for determining the presence of germ-line stem cells in mammals.

Yet another object of the present invention is to provide a method for diagnosing the differentiation ability of mammalian germ-line stem cells.

To achieve the above-mentioned objects of the present invention, in one aspect, the present invention provides a method for in vitro isolating and proliferating germ-line stem cells of mammals, the method comprising the step of culturing seminiferous tubules isolated from mammalian testis in an embryonic stem cell culture medium.

In another aspect, the present invention provides a method for in vitro differentiating germ-line stem cells, the method comprising the steps of encapsulating the germ-line stem cells and Sertoli cells of mammals with calcium alginate and three-dimensionally culturing the encapsulated cells.

In still another aspect, the present invention provides germ-line stem cells obtained by any one of said methods.

In still another aspect, the present invention provides a method for treating male infertility, the method comprising administering an effective amount of said germ-line stem cells to a subject.

In still another aspect, the present invention provides a composition for the treatment of male infertility, comprising said germ-line stem cells.

In still another aspect, the present invention provides a method for determining the presence or absence of germ-line stem cells in mammals.

In yet another aspect, the present invention provides a method for diagnosing the differentiation ability of mammalian germ-line stem cells. Hereinafter, the present invention will be described in detail.

The present invention relates to a method for isolating and proliferating germ-line stem cells, the method comprising the step of culturing a suspension of cells isolated from mammalian testis in an embryonic stem cell culture medium.

Specifically, the inventive method for the isolation and proliferation of germ-line stem cells comprises the steps of: (a) extracting testicular tissue from mammals; b) subjecting the extracted testicular tissue to a two-step enzymatic digestion process so as to prepare a cell suspension; and c) culturing the cell suspension in an embryonic stem cell culture medium.

The step of extracting the testicular tissue from mammals can be performed by a conventional method. For example, it can be performed by extracting a whole or a part of testis from mammals, washing the extracted tissue with PBS, and removing tunica albuginea from the washed tissue.

The two-step enzymatic digestion process can be performed by a known method (for example, Ogawa et al., Int. J. Dev. Biol., 41:111-122, 1997) or a modification thereof. For example, the enzyme solution used in said process may be a Ca²⁺ and Mg²⁺-free PBS (Phosphate Buffer Saline) supplemented with collagenase (Type I), DNase I, soybean trypsin inhibitor and hyaluronidase (Sigma).

The embryonic stem cell culture medium used in the present invention includes all media which may be used for the proliferation of embryonic stem cells. The embryonic stem cell culture medium is a DMEM (Dulbecco's modified Eagle's medium; GIBCO) supplemented with fetal bovine serum, nonessential amino acid, 2-mercaptoethanol, human leukemia inhibitory factor, bFGF and forskolin. More specifically, the embryonic stem cell culture medium used in the present invention may be a DMEM (Dulbecco's modified Eagle's medium; GIBCO) supplemented with 15% fetal bovine serum (HyClone), 1% nonessential amino acid (GIBCO), 10 μM 2-mercaptoethanol, 1500 U/ml of a human leukemia inhibitory factor (ESGRO), 1 ng/ml of bFGF (R&D) and 10 μM forskolin (Sigma).

The mammals in the present invention include all mammals, such as human beings, mice and cattle, and human beings are particularly preferred.

The inventive method for the isolation and proliferation of germ-line stem cells may additionally comprise the steps of treating the cells cultured in the embryonic stem cell culture medium with an enzyme so as to separate the cells into single cells and subculturing the single cells. The subculture medium has the same composition as that of the embryonic stem cell culture medium, and the subculture can be performed 5-7 times. The enzyme used in the enzyme treatment may be trypsin.

The inventive method for the isolation and proliferation of germ-line stem cells may additionally comprise the steps of mechanically sectioning the cells cultured in the embryonic stem cell culture medium and subculturing the cell sections in a prepared feeder layer. The feeder layer may be prepared by excluding germ line stem cells from the intratesticular peritubular cells cultured in the embryonic stem cell culture medium.

In one embodiment of the present invention, a cell suspension isolated from mouse testis was cultured in the embryonic stem cell culture medium according to the inventive isolation and proliferation method.

After 3-5 days of the culture, a number of multicellular colonies begun to be formed (see FIGS. 1A to 1C). Also, when cells isolated from the colonies were subcultured, colonies were formed again. The observation of the colonies subcultured five times showed that fibroblast-like cells were adhered to the bottom, on which multi-layer cell colonies were present (see FIGS. 1D and 1E). CD29, CD49f and Oct-4, markers expressed specifically in germ-line stem cells, were strongly expressed in the colonies (see FIGS. 1F to 1H). Also, SSEA-1, SSEA-3 and SSEA-4, which are expressed specifically in embryonic stem cells, were strongly expressed in the colonies (see FIG. 2).

These results suggest that when the cells isolated from mouse testis are cultured in the embryonic stem cell culture medium, colonies consisting of only germ-line stem cells can be formed, and thus, the isolation and proliferation of germ-line stem cells according to the present invention can be very effectively achieved at the same time.

In another embodiment of the present invention, a cell suspension isolated from non-obstructive azoospermia patients was cultured in the embryonic stem cell culture medium according to the inventive method for the isolation and proliferation of germ-line stem cells.

After 2-4 weeks of the culture, a number of multicellular colonies begun to be formed. When the cells isolated from the produced colonies were subcultured, colonies were then formed again. In case of the cells subcultured three times, colonies were also formed (see FIG. 3A). Alkaline phosphatase (see FIG. 3B), integrin β1 (see 3C) and integrin α6 (not shown), markers expressed specifically in germ-line stem cells, were strongly expressed in the produced colonies. Furthermore, from the fact that the mRNA bands of stem cell-specific Oct-4 and spermatogonia-specific c-kit were not detected before the culture but were detected after the culture, it could be found that the proliferation of germ-line stem cells was effectively achieved by the culture method used in the present invention (see FIG. 4).

These results indicate that when the cells isolated from non-obstructive azoospermia patients are cultured in the embryonic stem cell culture medium, colonies consisting of only germ-line stem cells can be formed, and thus, the inventive method can very effectively achieve the isolation and proliferation of germ-line stem cells at the same time.

Furthermore, the present invention relates to a method for the in vitro differentiation of germ-line stem cells, the method comprising the steps of encapsulating the germ-line stem cells and Sertoli cells isolated from mammalian testis with calcium alginate and three-dimensionally culturing the encapsulated cells.

The step encapsulating the cells with calcium alginate can be performed by a known method (for example, Lee et al., Biol. Reprod. 65: 873-878, 2001).

The culture medium for the differentiation of the encapsulated cells includes all differentiation media which can be used in the differentiation of germ-line stem cells. Specifically, the differentiation medium is a HEPES-buffered DMEM/F12 medium supplemented with insulin-transferrin-selenium solution, vitamin C, vitamin E, retinoic acid, retinol, pyruvate, recombinant human FSH, testosterone, antibiotic-antimycotic (ABAM) (containing penicillin, streptomycin and amphotericin B) and BCS. More specifically, it may be a HEPES-buffered DMEM/F12 medium supplemented with 10 μg/ml of insulin-transferrin-selenium solution, 10⁻⁴ M vitamin C, 10 μg/ml of vitamin E, 3.3×10⁻⁷ M retinoic acid, 3.3×10⁻⁷ M retinol, 1 mM pyruvate, 2.5×10⁻⁵ U recombinant human FSH, 10⁻⁷ M testosterone, 1× antibiotic-antimycotic (ABAM) (containing penicillin, streptomycin and amphotericin B) and 10% BCS.

In the three-dimensional culture of germ-line stem cells, it is preferred to co-culture the encapsulated cells with peritubular cells isolated from mammalian testis.

The peritubular cells are preferably isolated from the same mammalian species as that of the co-cultured germ-line stem cells. The peritubular cells may be isolated from mammals by a conventional method. Specifically, the peritubular cells may be prepared by isolating testicular cells from mammalian testis and culturing the isolated cells in a DMEM/F12 medium supplemented with bovine calf serum, FSH and testosterone. More specifically, they can be prepared by culturing the isolated cells in a DMEM/F12 medium supplemented with 10% bovine calf serum, 10 ng/ml of FSH and 10⁻³ M testosterone.

The germ-line stem cells and Sertoli cells of mice or human were encapsulated with calcium alginate and then cultured in the differentiation medium in one embodiment of the present invention, according to the inventive method for the in vitro differentiation of germ-line stem cells.

In one test example of the present invention, in order to examine the production of acrosome, the characteristics of round spermatids, a staining substance specifically binding to the acrosome was used. As a result, it could be found that the germ-line stem cells cultured according to the inventive method had acrosome which is the characteristics of spermatids (see FIG. 5).

In another test example of the present invention, the expression of c-kit known as a marker of spermatogonia and spermatocytes was examined by immunocytochemistry. The results showed that the c-kit was expressed not in the testicular cells isolated and proliferated from mice (see FIGS. 6A and 6C) but in the cells cultured according to the inventive method (see FIG. 6D). These results suggest that mouse germ-line stem cells cultured according to the inventive method can be differentiated into spermatogonia or spermatocytes. These results were also confirmed in human germ-line stem cells (see FIG. 9).

In another test example of the present invention, in order to examine the level of differentiation based on culture time, the transcription of mRNA of germ-line stem cell-specific Oct-4, spermatogonia-specific c-kit, spermatocyte-specific TH2B and spermatid-specific TP-1 genes were examined by RT-PCR. The results showed that germ-line stem cells cultured for 3 weeks would be differentiated into spermatocytes, and the cells cultured for 6 weeks would be differentiated into spermatids (see FIG. 7).

In still another test example of the present invention, in order to examine the effect of peritubular cells on the differentiation of germ-line stem cells, the transcriptional level of mRNA of spermatocyte-specific TH2B and spermatid-specific TP-1 genes was examined. The result showed that TH2B expression was reduced and TP-1 expression was increased in the case of co-culture with the peritubular cells, as compared to the otherwise case (see FIG. 8). These results suggest that the co-culture with the peritubular cells increase the efficiency of differentiation from spermatocytes to spermatids.

From the above results, it can be found that the mammalian germ-line stem cells encapsulation with calcium alginate followed by co-culture with the peritubular cells in the differentiation medium would significantly improve the effect on the differentiation of germ-line stem cells.

In one example of the present invention, in order to examine whether the round spermatids isolated, proliferated and differentiated according to the inventive method are fertilized with oocytes, the mouse round spermatids prepared according to the inventive method were injected into mature oocytes, and then, it was examined whether the division of embryos, the formation of blastocysts and the development of hatching were happened (see FIG. 10). The results showed that the embryos injected with the spermatids produced by the inventive method had no difference in the division level from a control group subjected only to activation but the embryos were higher in the level of blastocyst formation and the level of hatching than the control group. These results suggest that the spermatids produced by the inventive method have an excellent fertilizing capacity.

Furthermore, the present invention relates to germ-line stem cells obtained by the inventive isolation and proliferation method and to spermatids obtained by the inventive differentiation method.

Also, the present invention relates to a method for treating male infertility with germ-line stem cells obtained by the inventive isolation and proliferation method or spermatids obtained by the inventive differentiation method.

The method for treating male infertility with the spermatids may comprise the steps of in vitro fertilizing the spermatids with oocytes and implanting the embryos into the uterus. Specifically, the oocytes are collected by aspirating follicles more than 18 mm in diameter induced by ovulation. The aspirated oocytes are examined for their maturity and precultured depending on their maturity. Typical mature oocytes are precultured for 4-6 hours. In this regard, the maturity of oocytes is determined on the basis of the expansion of cumulus cells and the presence of a first polar body. One round spermatid can be aspirated into an injection pipette and then injected into a mature oocyte. Fertilized eggs are further cultured in a fresh culture medium for 24-48 hours and implanted into the uterus at the 4-6 cell stages. In embryo implanting, a special catheter inserted into the uterus may be used.

Also, the present invention relates to a composition for the treatment of male infertility containing germ-line stem cells obtained by the inventive isolation and proliferation method or spermatids obtained by the inventive differentiation method.

Male infertility in the present invention includes, but is not limited to, oligozoospermia, asthenozoospermia, teratozoospermia and azoospermia. The oligozoospermia means that the number of sperm per ml of semen is less than 20,000,000, the asthenozoospermia means that less than 50% of the total sperm have mobility, the teratozoospermia means that sperm with a normal shape are less than 50% as observed with a high-magnification microscope, and the azoospermia means that ejaculated semen has no sperm due to the obstruction of a vas deferense (obstructive azoospermia) or the insufficient production of sperm (non-obstructive azoospermia). The azoospermia is preferably non-obstructive azoospermia.

Furthermore, the present invention relates to a method for determining the presence of germ-line stem cells in mammals, the method comprising the steps of: (a) extracting testicular tissue from mammals; (b) subjecting the testicular tissue to a two-step enzymatic digestion process so as to prepare a cell suspension; and (c) culturing the cell suspension in an embryonic stem cell culture medium and then determining whether cell colonies were formed.

In addition, the present invention relates to a method for diagnosing the differentiation ability of germ-line stem cells, the method comprising the steps of: (a) extracting testicular tissue from mammals; (b) subjecting the testicular tissue to a two-step enzymatic digestion process so as to prepare a cell suspension; (c) culturing the cell suspension in a stem cell culture medium; and (d) encapsulating the cultured germ-line stem cells and Sertoli cells with calcium alginate followed by three-dimensionally culturing the encapsulated cells. In this regard, the mammals designate mice or human beings, particularly human patients with male infertility.

### BRIEF DESCRIPTION OF THE DRAWINGS

FIG. 1 shows microscopic photographs showing the results of immunocytochemistry and in situ hybridization for cells isolated and proliferated from mouse testis.

A: initial stage of culture; B: an enlarged photograph of A (after 3 days of culture); C: AP/DAPI staining; D: AP (100×); E: an enlarged photograph of D (200 X); F: CD29; G: CD49f; and H: Oct-4. FIGS. 1A to 1C show cells in the initial stage of culture, and FIGS. 1D to 1H show cells subcultured five times.

FIG. 2 shows microscopic photographs showing the results of immunocytochemistry for embryonic stem cell markers on cells isolated and proliferated from mouse testis.

A: SSEA-1; B: SSEA-3; and C: SSEA-4.

FIG. 3 shows microscopic photographs showing the results of immunocytochemistry for germ-line stem cell markers on cells isolated and proliferated from the testis of human non-obstructive azoospermia patients.

A: no staining; B: AP staining; C: Integrin β1; and D: Hoechst nucleic staining.

FIG. 4 shows an electrophoresis photograph showing the results of RT-PCR to examine the mRNA transcription pattern of spermatogenesis-specific genes of cells isolated and proliferated from human non-obstructive azoospermia patients.

Lane 1: fertile man; lane 2: man with no germ cell (negative control); lane 3: man 1 with no germ-line stem cells (GSCs)+0 week; lane 4: man 1 with no GSCs+culture for 2 weeks; lane 5: man 2 with GSCs+0 week; lane 6: man 2 with GSCs+culture for 2 weeks; and N: no loading.

FIG. 5 shows microscopic photographs showing the results of TRITC-PNA staining to examine the acrosome of cells isolated, proliferated and differentiated from mouse testis.

A: optical microscopic photograph; and B: fluorescent microscopic photograph. Fluorescence intensity was more intense in the acrosomal region (arrow).

FIG. 6 shows microscopic photographs showing the expression of c-kit to examine the differentiation of cells isolated and proliferated from mouse testis, before and after culture.

A: testis of a 5-day-old mouse before culture; B: testicular cells of a 15-day-old mouse before culture; C: testicular cells of a 5-day-old mouse before culture; and D: testicular cells of a 5-day-old mouse after 3 weeks of culture.

FIG. 7A shows an electrophoresis photograph showing the results of RT-PCR to examine the mRNA transcription pattern of spermatogenesis-specific genes of germ-line stem cells isolated and proliferated from mouse testis.

Lane 1: a 5-day-old mouse; lane 2: a 10-day-old mouse; lane 3: a 15-day-old mouse; lane 4: a 20-day-old mouse; and lane 5: an adult mouse.

FIG. 7B shows an electrophoresis photograph showing the results of RT-PCR to examine the mRNA transcription pattern of spermatogenesis-specific genes of germ-line stem cells isolated, proliferated and differentiated from the testis of a neonatal mouse.

Lane 1: before culture; lane 2: differentiation by 3-week culture; lane 3: differentiation by 6-week culture; and N: no loading.

FIG. 8 shows an electrophoresis photograph showing the results of RT-PCR to examine the effect of the co-culture with peritubular cells on the differentiation of mouse germ-line stem cells.

Lane 1: before culture; 2: 1-week culture after encapsulation of the cells with calcium alginate; lane 3: 1-week culture; lane 4: 1 week co-culture with peritubular cells after encapsulation of the cells with calcium alginate; and NC: negative control.

FIG. 9A shows a photograph showing the results of RT-PCR analysis to examine the expression pattern of Oct-4 genes in cells isolated and proliferated from 6 human non-obstructive azoospermia patients. Three of the patients had germ-line stem cells (lanes 1, 2 and 5), and the others had no germ-line stem cells (lanes 3, 4 and 6).

FIG. 9B shows a photograph showing the results of RT-PCR to examine the expression pattern of Oct-4, c-kit and TP-1 genes in cells isolated from one patients (lane 5) having germ-line stem cells in FIG. 9A and cultured according to the inventive proliferation method for 2 weeks.

FIG. 10 shows photographs showing the production of embryos are produced when round spermatids isolated, proliferated and differentiated according to the inventive method are injected into oocytes.

A: cultured germ-line stem cells following encapsulation with calcium alginate; B: separation of differentiated round spermatids; C: oocytes injected with round spermatids; and D: blastocysts generated with oocytes injected with round spermatids.

### BEST MODE FOR CARRYING OUT THE INVENTION

Hereinafter, the present invention will be described in detail by examples. It is to be understood, however, that these examples are provided for illustrative purpose only and are not construed to limit the scope of the present invention.

### EXAMPLE 1

### Isolation and Proliferation of Germ-Line Stem Cells from Male Mice

Germ-line stem cells were isolated from the testis of thirty 3˜5-day-old male C57BL/6 or ICR mice (Korea Biolink Co.) and proliferated.

First, in order to isolate seminiferous tubules from male mice, the following two-step enzymatic digestion process (Ogawa et al., Int. J. Dev. Biol., 41:111-122, 1997) was performed.

Namely, testis were extracted from the mice and washed with PBS, and the tunica albuginea was removed from the testis. Seminiferous tubules, which are exposed in the testis, were collected and incubated in 10 ml of a first enzyme solution containing 0.5 mg/ml of collagenase (Type I, Sigma), 10 μg/ml of DNase I, 1 μg/ml of soybean trypsin inhibitor (Gibco, Grand Island, N.Y.), 1500 U/ml of leukemia inhibitory factor (ESGRO) and 1 mg/ml of hyaluronidase (Sigma) in Ca²⁺ and Mg²⁺-free PBS at room temperature for 20 minutes. Then, PBS was added to the first enzyme solution and it was centrifuged at 1,500 rpm for 4 minutes. The supernatant was removed and pellet was obtained.

The peritubular cells were removed by the above process, and then the pellet was incubated in 10 ml of a second enzyme solution containing 5 mg/ml of collagenase (Type I, Sigma), 10 μg/ml of DNase I, 1 μg/ml of soybean trypsin inhibitor (Gibco, Grand Island, N.Y.) and 1 mg/ml of hyaluronidase (Sigma) in Ca²⁺ and Mg²⁺-free PBS at 37° C. for 30 minutes. Then, PBS was added to the second enzyme solution and it was centrifuged at 1,500 rpm for 4 minutes. The supernatant was removed and pellet was obtained.

The pellet was attached to a culture dish coated with 0.2% gelatin and cultured. The culture medium was DMEM (Dulbecco's modified Eagle's medium; GIBCO) supplemented with 15% fetal bovine serum (HyClone), 1% nonessential amino acid (GIBCO), 10 μM 2-mercaptoethanol, 1500 U/ml of leukemia inhibitory factor (ESGRO), 1 ng/ml of bFGF (R&D) and 10 μM forskolin (Sigma). Also, the pellet was cultured in an incubator maintained at 5% CO₂ and 95% humidity. After 3-5 days culturing, large multi-cellular colonies were formed.

In order to subculture, the produced germ-line stem cell colonies were treated with 0.25% trypsin at 37° C. for 6 minutes at six-day intervals, and single cells were isolated from the culture dish using a pipette, and a serum-containing medium was added in order to inhibit trypsin reaction. Then, the culture solution was centrifuged at 1,500 rpm for 4 minutes. The supernatant was removed and pellet was obtained.

The pellet was resuspended in a medium having the same composition as that of said medium, and cultured in a fresh culture dish. It was observed that the isolated single cells were able to form colonies again when cultured.

### TEST EXAMPLE 1

### Examination of Isolation and Proliferation of Mouse Germ-Line Stem Cells

In order to examine whether the colonies formed in Example 1 are germ-line stem cells, immunocytochemistry and in situ hybridization were performed.

(1-1) Examination of Expression of Germ-Line Stem Cell Markers

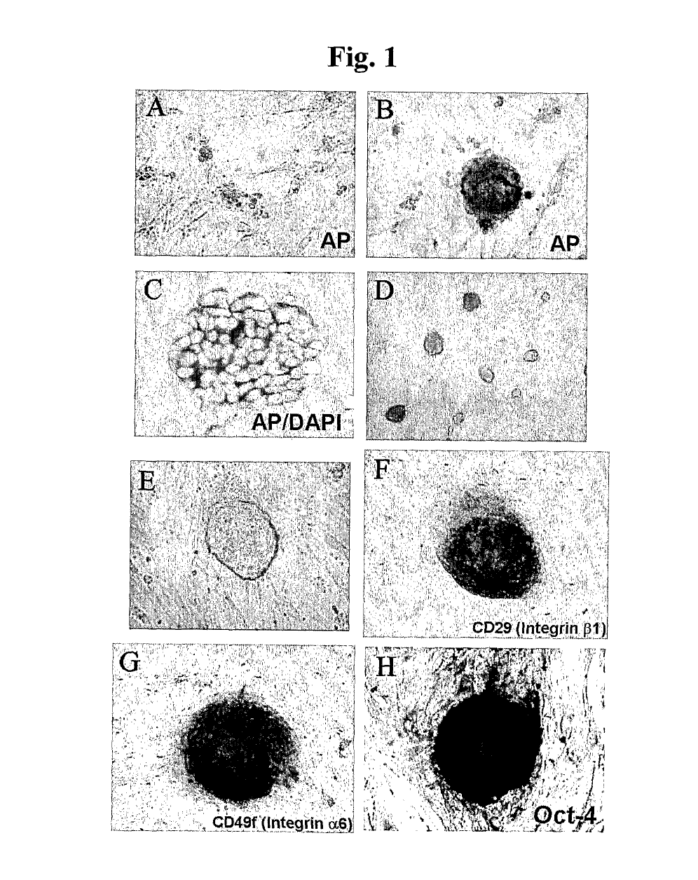

The expression of alkaline phosphatase (AP) known as a germ-line stem cell marker was examined. Namely, the cells of the colonies produced by six-day culture and 5-times subculture in Example 1 were fixed in 66% acetone/3.7% formaldehyde. Then they were stained with a naphtol/FRV-alkaline AP substrate (Sigma) or DAPI (nucleus staining substance; FIG. 1C) and observed.

The expression of other germ-line stem cell markers, Integrin α6 chain and Integrin β1 chain, was examined by immunocytochemistry. Namely, the cells of the colonies produced in Example 1 were fixed with 4% paraformaldehyde (GIBCO/BRL) in Dulbecco's PBS. Primary antibody was then added at a concentration of 1:500. The cell was incubated at ambient temperature for 2 hours. The cell was added to a secondary antibody and incubated. As the primary antibodies for immunocytochemistry, CD49f (Integrin α6 chain; BD/Pharmingen) and CD29 (Integrin β1 chain; BD/Pharmingen) were used. As the secondary antibody for the detection of the primary antibodies, biotinylated anti-mouse IgM antibody, ABC-AP (Vector Lab. Inc.), was used. A mixture of BCIP (Sigma) and NBT (Sigma) in AP reaction buffer was used for a color development.

FIG. 1 shows microscopic photographs of said cells. As shown in FIG. 1, it was observed that the cells strongly expressing AP were attached and aggregated on the culture dish after 6-day culturing (see FIG. 1A). From an enlarged view of FIG. 1, it could be seen that a number of the cells were proliferated in the form of colony (see FIGS. 1B and 1C). After five times subculturing, fibroblast-like cells adhered to the bottom and multi-layer cell colonies were observed on the fibroblast-like cells (see FIG. 1D). The cells adhered to the bottom were presumed as Sertoli cells, and the colony cells thereon were presumed as germ-line stem cells. From an enlarged view of one of the colonies presumed as germ-line stem cells, it could be seen that the one colony consisted of a mass of several cells (see FIG. 1E).

Also, the expression of Oct-4 known as another marker of embryonic stem cells was examined by in situ hybridization. First, the cells of the colonies produced by six-day culturing and five-times subculturing in Example 1 were fixed with MEMFA (0.1 M MOPS, pH 7.5, 2 mM EDTA, 1 mM MgSO₄, 3.7% formaldehyde) at ambient temperature for 20 minutes. The hybridization was performed in the same manner as the known in situ hybridization method (Song et al., Dev. Biol., 213:157-169, 1999) except that the hybridization and subsequent washing processes were conducted at 55° C. As a result, Oct-4 which is expressed specifically in embryonic stem cells was strongly expressed only in the colonies presumed as germ-line stem cells (FIG. 1H).

From the above results, it can be found that when the cells isolated from mouse testis as described above in Example 1 are cultured in a simple medium used for the proliferation of embryonic stem cells, the germ-line stem cells will form colonies. This indicates that the isolation and proliferation of the mouse germ-line stem cells can be very effectively achieved at the same time.

(1-2) Examination of Expression of Embryonic Stem Cell Markers

The expression of SSEA (Stage Specific Embryonic Antigen)-1, SSEA-3 and SSEA-4 known as markers of embryonic stem cells and embryonic germ cells was examined in the same method as in Test Example 1-1, except that MC480 (SSEA-1), MC631 (SSEA-3) and MC813-70 (SSEA-4) antibodies (Developmental Studies Hybridoma Bank, University of Iowa) were used as primary antibodies for immunocytochemistry.

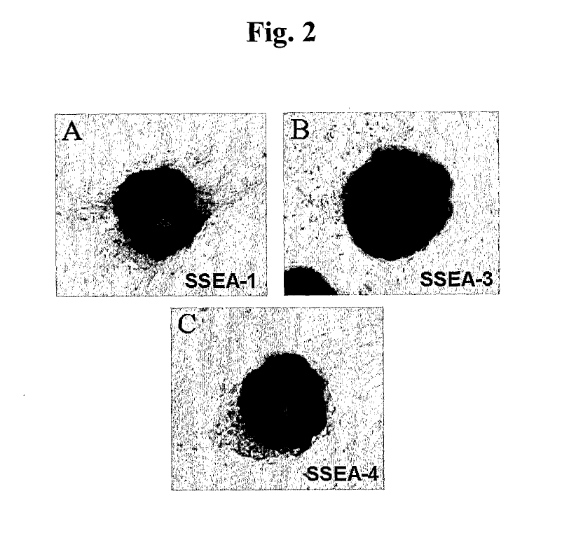

FIG. 2 shows microscopic photographs of said cells. As shown in FIG. 2, SSEA-1, SSEA-3 and SSEA-4, which are expressed specifically in the embryonic stem cells, were strongly expressed only in the colonies presumed as germ-line stem cells (see FIGS. 2A to 2C). These results suggest that the cells cultured as described in Example 1 also have the characteristic of mouse embryonic stem cells.

### EXAMPLE 2

### Isolation and Proliferation of Germ-Line Stem Cells from Human Azoospermia Patients

Germ-line stem cells were isolated from the testis of 13 human non-obstructive azoospermia patients and proliferated.

First, in order to isolate seminiferous tubules from the 13 patients, a portion of the testicular tissue was extracted by biopsy and subjected to the two-step enzymatic digestion process in the same manner as in Example 1.

The pellets produced by above method were attached and cultured on a culture dish coated with 0.2% gelatin. A culture medium was DMEM (Dulbecco's modified Eagle's medium; GIBCO) supplemented with 15% fetal bovine serum (HyClone), 1% nonessential amino acid (GIBCO), 10 μM 2-mercaptoethanol, 1500 U/ml of human leukemia inhibitory factor (ESGRO), 4 ng/ml of bFGF(R&D) and 10 μM forskolin (Sigma). Also, the culture was performed in an incubator maintained at 5% CO₂ and 95% humidity. After 2-4 week culturing, a number of multicellular colonies and Sertoli cell-similar feeder cells were formed in six samples (46.2%) among the 13 patients. For subculture at 2-week intervals, the colonies on the feeder cells were mechanically disaggregated, and dissociated into three pieces using a micropipette, and then transferred to another feeder cells. Other conditions of the subculture were the same as in Example 1. The colonies transferred to other feeder cells were successfully attached and proliferated until 6-7^{th} passage. It was observed that the isolated cells were able to form colonies again when cultured. Also, in the samples of other three patients, colony-like structures were formed in other three patients, but disappeared after passages.

### TEST EXAMPLE 2

### Examination of Isolation and Proliferation of Human Germ-Line Stem Cells

In order to examine whether the colonies isolated and proliferated in Example 2 are germ-line stem cells, immunocytochemistry and RT-PCT (reverse transcription-polymerase chain reaction) were performed.

(2-1) Examination of Expression of Germ-Line Stem Cells Markers

In order to examine whether AP (alkaline phosphatase), CD49f (Integrin α6 chain) and CD29 (Integrin β1 chain) known as germ-line stem cell markers are expressed in the cells formed in three times-subculture, the immonocytochemistry was performed according to the method described in test example 1-1.

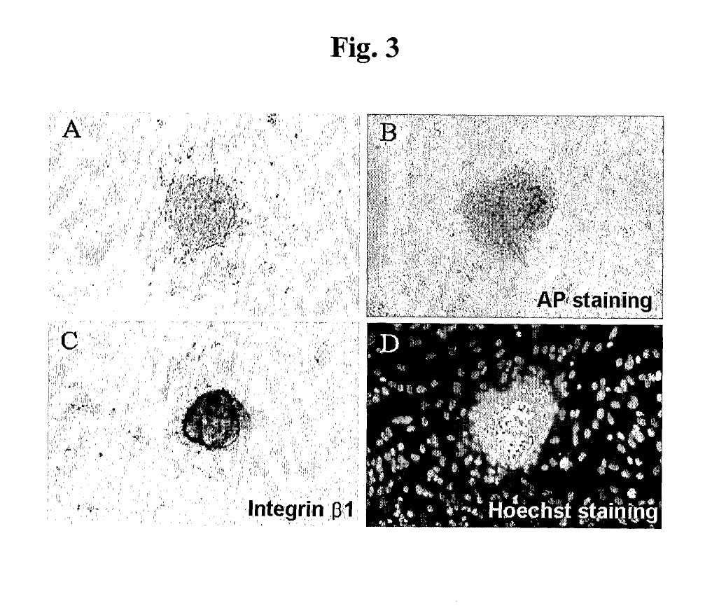

FIG. 3 shows microscopic photographs of the stained cells. As shown in FIG. 3, AP (FIG. 3B), CD29 (FIG. 3C) and CD49f (data not shown), which are expressed specifically in germ-line stem cells, were strongly expressed in the colonies.

From these results, it can be found that when the seminiferous tubules isolated from the testis of human non-obstructive azoospermia patients are cultured in a medium for the proliferation of embryonic stem cells as described in Example 2, the germ-line stem cells will form colonies. This indicates that the isolation and proliferation of the human germ-line stem cells can be very effectively achieved at the same time.

(2-2) Examination of Expression of Spermatogenesis-Specific Genes

In order to examine the characteristics of the cells cultured in Example 2, the mRNA transcription pattern of spermatogenesis-specific genes was analyzed by the RT-PCR. As the spermatogenesis-specific genes, Oct-4 (germ-line stem cell-specific), c-kit (spermatogonia- and spermatocyte-specific) and TP-1 (spermatid-specific) were used.

This Test Example was performed for the following testicular cells: testicular cells isolated from a fertile man; testicular cells isolated from a man with no germ cell, as a negative control; testicular cells isolated from man 1 with no GSCs; testicular cells isolated from man 1 with no GSCs and cultured for 2 weeks as described in Example 2; testicular cells isolated from man 2 with GSCs; and testicular cells isolated from man 2 with GSCs and cultured for 2 weeks as described in Example 2.

First, each of the above cells was incubated with trypsin-EDTA for 30 minutes, washed three times with Ca²⁺ and Mg²⁺-free PBS and sampled. About 100 mg of total RNA of the above testis and cultured cells was extracted according to the TRIzol method (Gibco).

According to a method described in Huang et al., Biotechniques 20: 1012-1020, 1996, the reverse transcription of RNA was performed by following procedures: 1 μg of total RNA, 5 mM MgCl₂ and 1U DNase I mixed and reacted at 37° C. for 30 minutes followed by adding 1 mM dNTP, 2.5 mM oligo dT and 2.5 U reverse transcriptase (SuperScript, Gibco), and reacting at 42° C. for 1 hour. The resulting cDNA was used as a template for PCR.

PCR was performed for Oct-4, c-kit and TP-1 genes, as well as 18S ribosomal protein as a positive control. The PCR reaction for the genes was performed in 20 μl of a reaction solution containing 3-5 μmol of each of primers having sequences shown in Table 1 below, 10 mM Tris-HCl (pH 8.3), 2 mM MgCl₂, 50 mM KCl, 0.25 mM dNTP and 1.25 U Taq polymerase (Gibco). The PCR reaction consisted of initial denaturation of 5 minutes at 94° C., and then 35 cycles of 30 seconds at 94° C., 30 seconds at 60° C. and 30 seconds at 72° C., followed by final extension of 10 minutes at 72° C. The PCR product was separated by 2% agarose gel electrophoresis.

TABLE 1

| Gene | Primer sequence | Source |
|---|---|---|
| Oct-4 | 5′-acc atg ttt ctg aag tgc cc-3′ | GeneBank X52437 |
| | (forward, SEQ ID NO: 1) | |
| | 5′-gct cct gat caa cag cat ca-3′ | |
| | (reverse, SEQ ID NO: 2) | |
| c-kit | 5′-ctg gtg gtt cag agt tcc ata gac-3′ | GeneBank Y00864 |
| | (forward, SEQ ID NO: 3) | |
| | 5′-tca acg acc ttc ccg aag gca cca-3′ | |
| | (reverse, SEQ ID NO: 4) | |
| TP-1 | 5′-tgg cat gag gag agg caa ga-3′ | GeneBank NM009407 |
| | (forward, SEQ ID NO: 5) | |
| | 5′-gct cat tgc cgc atc aca ag-3′ | |
| | (reverse, SEQ ID NO: 6) | |
| 18S | 5′-aga tga tcg agc cgc gc-3′ | Wagner and Perry Mol |
| ribosomal | (forward, SEQ ID NO: 7) | Cell Biol 5: |
| protein | 5′-gct acc agg gcc ttt gag atg ga-3′ | 3560-3576, 1985 |
| | (reverse, SEQ ID NO: 8) | |

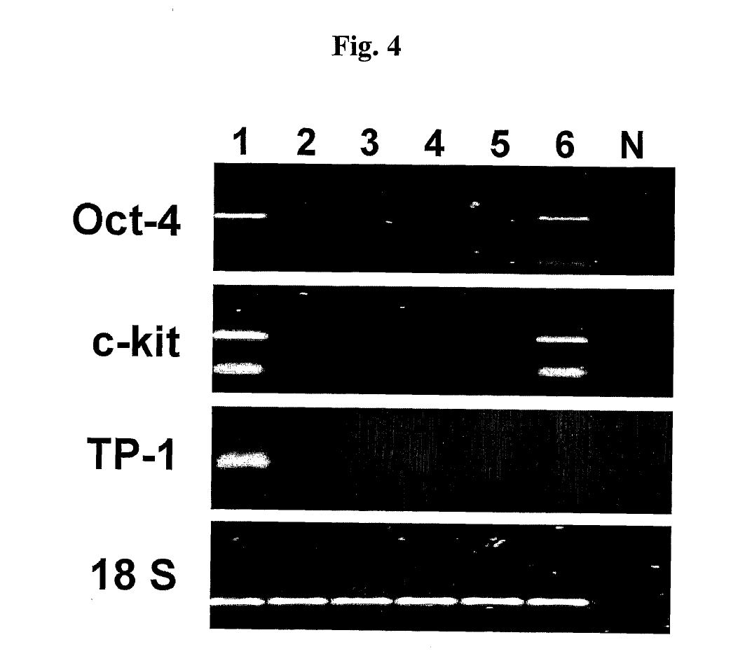

The results were shown in FIG. 4. As shown in FIG. 4, the mRNA bands of Oct-4 and c-kit were not detected (lane 5) before culturing isolated cells as described in Example 2, whereas the mRNA bands of Oct-4 and c-kit were detected (lane 6) after culturing.

From these results, it could be found that even when the testicular tissue of human non-obstructive azoospermia patients contained a very small number of germ-line stem cells as in Example 2, the germ-line stem cells would be successfully proliferated in vitro according to the inventive method.

### EXAMPLE 3

### In Vitro Differentiation from Germ-Line Stem Cells into Haploid Germ Cells

The isolated and proliferated germ-line stem cells were differentiated into germ cells by in vitro culture.

Namely, the germ-line stem cells and Sertoli cells of mice and human non-obstructive azoospermia patients, which have been isolated and proliferated in Examples 1 and 2, respectively, were separated into single cells by treatment with trypsin, and resuspended, followed by encapsulation with sodium alginate (Lee et al., Biol. Reprod. 65: 873-878, 2001).

Then, the encapsulated cells were transferred to 1.0 ml of a culture medium without peritubular cell monolayers as feeder cells in 24-well dish, and cultured in an incubator (maintained at 5% CO₂ and 95% humidity) at 32° C. for 7 weeks. During culture, the culture medium was replaced every two day. The culture medium was a HEPES-buffered DMEM/F12 medium supplemented with 10 μg/ml of insulin-transferrin-selenium solution (Gibco), 10⁻⁴ M vitamin C (Sigma), 10 μg/ml of vitamin E (Sigma), 3.3×10⁻⁷ M retinoic acid (Sigma), 3.3×10⁻⁷ M retinol (Sigma), 1 mM pyruvate (Sigma), 2.5×10⁻⁵ U recombinant human FSH (Gonal-F; Serono), 10⁻⁷ M testosterone (Sigma), 1× antibiotic-antimycotic (ABAM) (containing penicillin, streptomycin and amphotericin B; Gibco) and 10% BCS (Weiss et al., Biol. Reprod. 57: 68-76, 1997) (single culture group). The concentrations of FSH and testosterone were increased during the culture period such that, after 7 weeks of the culture, the concentrations were increased up to 1,000 times for final differentiation.

Meanwhile, the testicular cells (including peritubular cells) of a 15-day-old mouse were attached and cultured on a culture dish. The culture medium was a DMEM/F12 medium supplemented with 10% bovine calf serum, 10 ng/ml of FSH and 10⁻³ M testosterone. After one week of culture, peritubular cell monolayers was formed and used as feeder cells. In order to examine the effect of the peritubular cells on the differentiation of germ-line stem cells, the peritubular cell monolayers were co-cultured with the encapsulated cells in a medium used in single culture group according to the manner as described above (co-culture group).

### TEST EXAMPLE 3

### Examination of Differentiation from Mouse Germ-Line Stem Cells into Germ Cells

In order to examine whether mouse germ-line stem cells are differentiated into germ cells by the method of Example 3 and to examine the extent of the differentiation, immunohistochemistry and RT-PCR (reverse transcription-polymerase chain reaction) were performed.

(3-1) Examination of Production of Acrosomes

In order to examine whether the acrosomes of round spermatids are produced, the cells of the single culture group were observed under a microscope.

Namely, the cells cultured in Example 3 were mechanically decapsulated and incubated in trypsin-EDTA for 30 minutes. Then, the dispersed cells were rinsed with PBS and fixed with 5% paraformaldehyde for 30 minutes at room temperature. The cells were washed three times and attached onto a precoated slide glass (Probe On Plus™, Fisher, Pa.) by cytospin (Cyto-TEK™, Miles Inc., Elkhart, Ind.) at 1,500 rpm for 15 minutes, and then permeated with anhydrous methanol for 30 minutes. The cells were reacted with 10 μg/ml of tetramethylrhodamine isothiocyanate-peanut agglutinin (TRITC-PNA) binding specifically to acrosome granules, at room temperature for 1 hour. After washed, the cells were transferred into a slide glass with PBS and covered with a cover glass. Then, the cells were sealed with nail vanish and observed under a microscope.

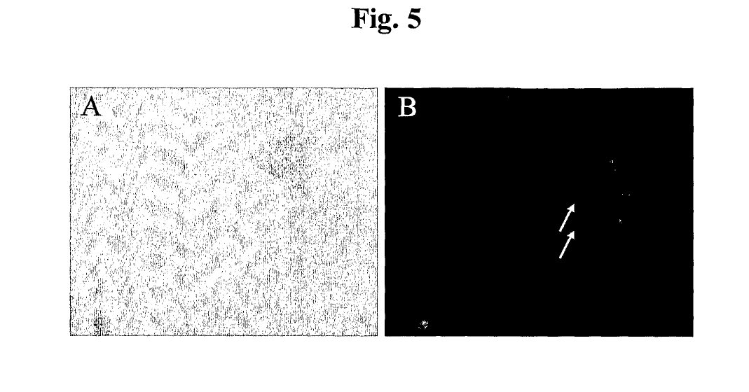

The results are shown in FIG. 5. As shown in FIG. 5, the cells subjected to the culture process of Example 3 had acrosome, which are the characteristics of round spermatids (arrow). These results suggest that germ-line stem cells are differentiated into round spermatids by the culture method described in Example 3.

(3-2) Examination of Expression of c-Kit

In order to examine the expression of c-kit known as a marker of spermatogonia and spermatocytes, the following test was performed.

Cell masses encapsulated with alginate were collected at 0, 3 and 6 weeks after culture, and each of the collected cell masses was fixed in PBS buffer supplemented with 10% formaldehyde for 24 hours. Then, each of the samples was washed with PBS for 24 hours, and dehydrated two times with varying concentrations (50, 70, 85, 96 and 100%) of ethanol for 20 minutes each time, cleaned two times with xylene for 30 minutes each time, infiltrated two times with paraffin at 60° C. for 30 minutes each time, and embedded into paraffin wax. Thereafter, continuous sections (5 μm) were prepared, dried on a slide glass at 37° C. overnight, and stored at room temperature until immunohistochemistry.

The prepared sections were deparaffinized with xylene and rehydrated with varying concentrations of ethanol. The sections on the slide glass were rinsed three times with a reaction buffer which has been reacted with an anti-c-kit antibody at 4° C. overnight. The detection of the primary monoclonal antibody was performed with a biotinylated secondary antibody and then with a mixture of streptavidin and horseradish peroxidase. Next, a DAB substrate-chromogen solution (Histostain-Plus DAB kit, Zymed, CA) was added in order to examine the presence of peroxidase.

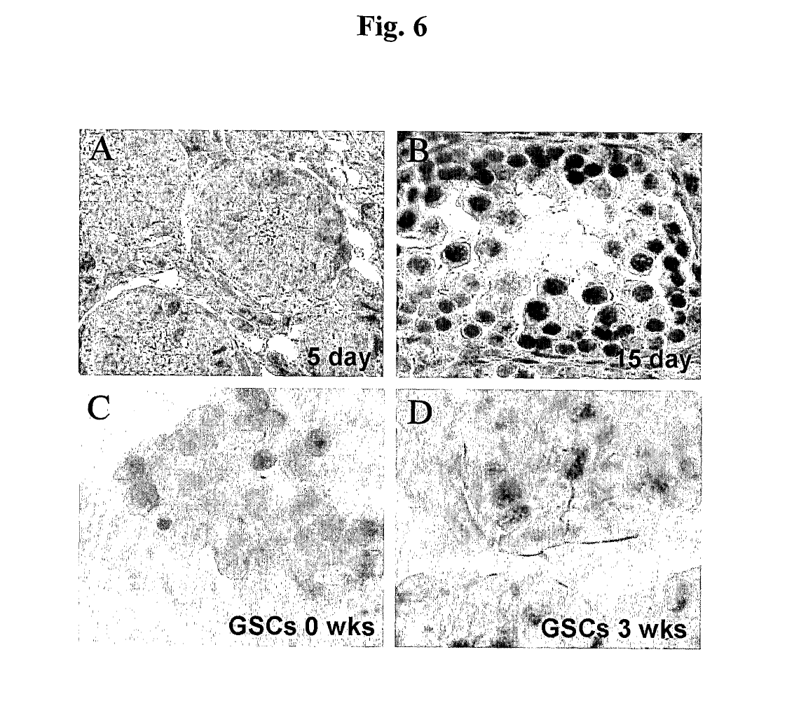

The results are shown in FIG. 6. As shown in FIG. 6, the expression of c-kit could not be observed in the testis of the 5-day-old mouse (FIG. 6A), whereas it could be observed in the testicular cells of the 15-day-old mouse (FIG. 6B). Meanwhile, the c-kit was expressed in the case of culturing the testicular cells isolated and proliferated from the 5-day-old mouse for 3 weeks in the same manner as in Example 3 (FIG. 6D). From these results, it can be found that germ-line stem cells will be differentiated into spermatogonia or spermatocytes in the case of culture as done for the single culture group of Example 3.

(3-3) Examination of Expression of Spermatogenesis-Specific Gene

In order to examine the characteristics of the cells cultured in Example 3, the mRNA transcription of Oct-4 (germ-line stem cell-specific), c-kit (spermatogonia-specific and spermatocyte-specific), TH2B (spermatocyte-specific) and TP-1 (spermatid-specific) genes was examined by RT-PCR. The examination was performed in the same manner as in Test Example 2-2 except for conditions specified below.

In order to examine the process of differentiation of germ-line stem cells with the passage of time after the birth of mice, the test was performed on germ cells isolated from 5-day-old, 10-day-old, 15-day-old and 20-day-old and adult mice. Also, germ-line stem cells isolated from the testis of neonatal mice were examined the cells cultured according to the method of Example 3 (0, 3 and 6 weeks). As primers for PCR, sequences set forth in Table 1 above and Table 2 below were used.

TABLE 2

| Gene | Primer sequence | Source |
|---|---|---|
| TH2B | 5′-ctc tct aga aag gtt act tga gcc atg-3′ | GeneBank X90778 |
| | (forward, SEQ ID NO: 9) | |
| | 5′-ctc tat aga tgc cgg tgt cgg ggt g-3′ | |
| | (reverse, SEQ ID NO: 10) | |

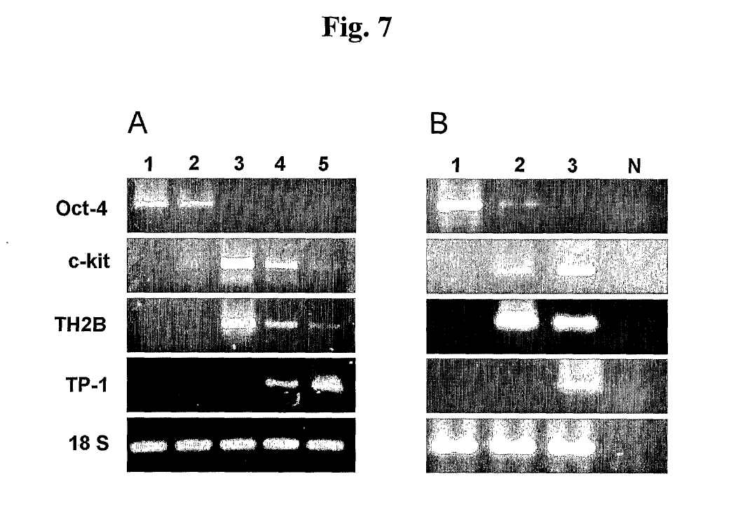

The results are shown in FIGS. 7A and 7B. From FIG. 7A, it could be found that, at 20 days after birth, the germ-line stem cells in the testis of wild-type mice were differentiated to spermatids via spermatogonia and spermatocytes. Also from FIG. 7B, it could be found that 3-week culture of the testicular cells of the neonatal mice containing only undifferentiated germ-line stem cells according to the method of Example 3, make the germ-line stem cells differentiate to spermatocytes in vitro, and 6-week culture make them differentiate to spermatids.

(3-4) Examination of Effect of Peritubular Cell on Differentiation

In order to examine the effect of the co-culture system designed in Example 3 on the differentiation of germ-line stem cells, real time-PCR was performed for TH2B (spermatocyte-specific) and TP-1 (spermatid-specific) genes, so as to measure the mRNA transcription and transcriptional level of the genes. Cells isolated from a 15-day-old mouse were used.

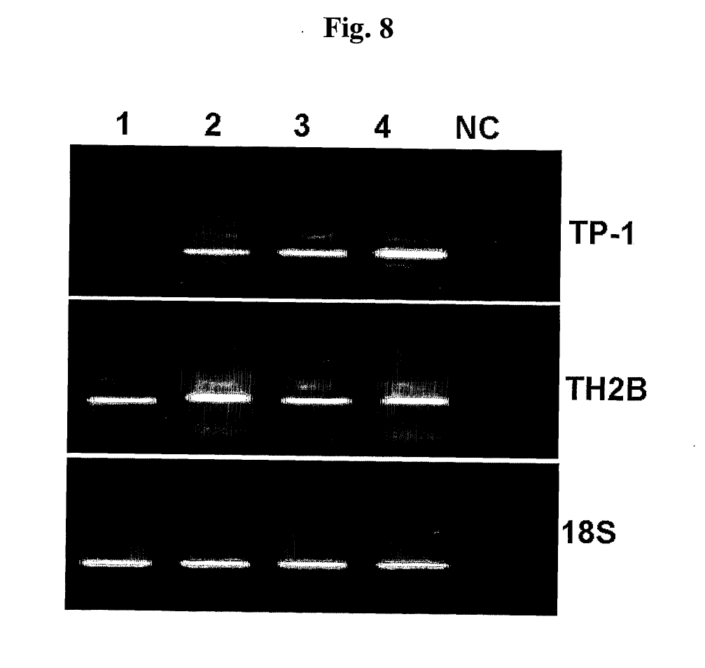

The results are shown in FIG. 8. As shown in FIG. 8, the expression of the spermatocyte-specific TH2B gene was decreased in the three-dimensional coculture with peritubular cells for one week (lane 4) as compared to the single culture (lane 2) and the general culture (lane 3), but the expression of the spermatid-specific TP-1 gene was increased. From these results, it could be found that the three-dimensional co-culture system with the use of peritubular cells (lane 4) was higher in the efficiency of differentiation from germ cells into spermatids than the single culture (lane 2) or the general culture (lane 3).

The results of Test Examples 3-1 to 3-4 indicate that pre-culture encapsulation of germ-line stem cells isolated and proliferated from mice with calcium alginate make the differentiation of them efficient, and the co-culture of the encapsulated germ-line stem cells with peritubular cells make the differentiation of them more efficient.

### TEST EXAMPLE 4

### Examination of Differentiation from Human Germ-Line Stem Cells into Germ Cells

In order to examine whether human germ-line stem cells are differentiated into germ cells by the method of Example 3, RT-PCR used in Examples above was performed for Oct-4 (germ-line stem cell-specific) and c-kit (spermatogonia-specific and spermatocyte-specific) genes. And the mRNA transcription of the genes was determined.

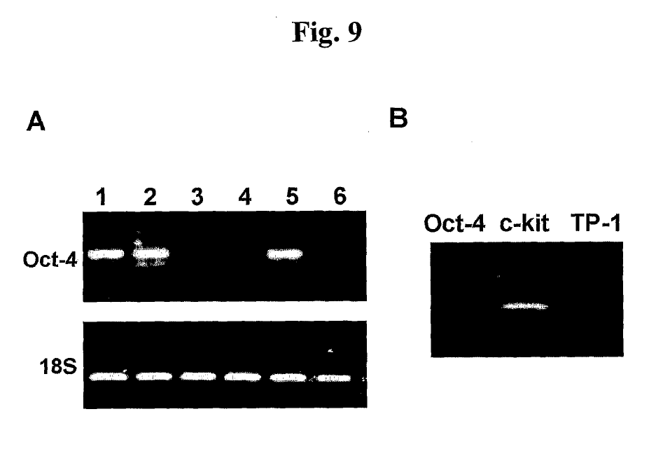

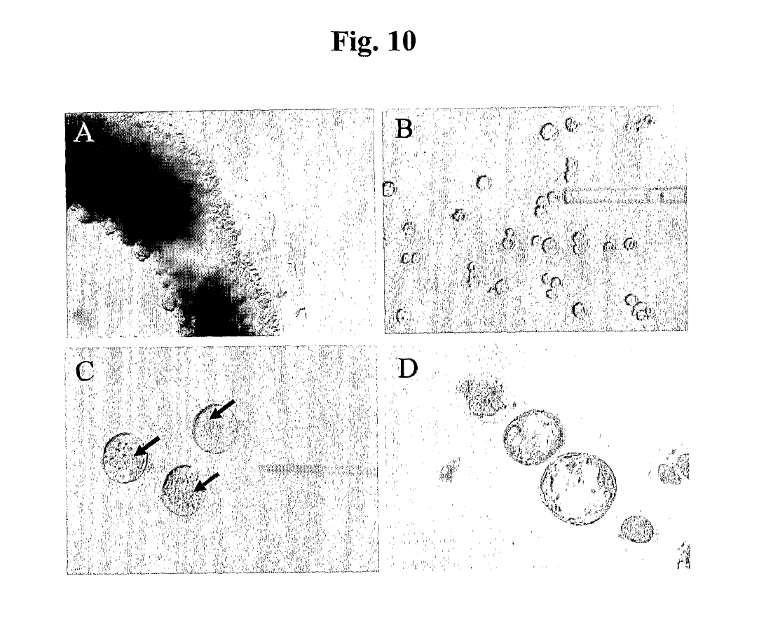

The results are shown in FIG. 9. As shown in FIG. 9, in the case where a suspension of cells extracted from the testicular tissue of six non-obstructive azoospermia patients was cultured, Oct-4 mRNA was expressed in patients (Nos. 1, 2 and 5) whose cells formed colonies and not expressed in patients (Nos. 3, 4 and 6) whose cells did not form colonies (FIG. 10A). These results suggest that the formation of colonies can provide a diagnosis of the presence of germ-line stem cells. Also, the colony cells of one of the patients (No. 5) together with supporting cells were encapsulated with calcium alginate and cultured for 2 weeks, and as a result, the mRNA of the c-kit gene, which is a marker of spermatogonia and spermatocytes, was expressed and the germ-line stem cells was differentiated into spermatogonia and spermatocytes (FIG. 9B).

### EXAMPLE 4

### Injection of Spermatids into Oocyte Cytoplasm and Examination of Formation of Embryos

In order to examine whether the germ-line stem cells isolated, proliferated and differentiated in the present invention are fertilized with oocytes, the mouse cells produced in Example 3, which have been confirmed to be round spermatids in Test Example 3, were isolated from the culture solution and injected into mature oocytes.

First, in order to obtain oocytes, expanded cumulus cells were removed by repeatedly pipetting with a small glass pipette, and the oocyte was incubated in 0.1 mg/ml of hyaluronidase for 5 minutes. Then, the mature oocyte was activated with 5 μM calcium ionophore (Sigma) and cultured for 3 hours.

One round spermatid selected for injection was aspirated with an injection pipette (FIGS. 10A and 10B), and injected into the pre-treated oocyte in a direction of 90° from the first polar body (FIG. 10C). The oocyte injected with the spermatid was transferred and cultured in a 0.3% BSA-containing KSOM medium (Lawitts et al., J Reprod Fertil 91:543-56, 1991) under mineral oil. The culture medium was replaced every two days. We evaluated the embryonic cleavage, the formation of blastocyst and hatching where the embryo comes out through the Zona Pellucida. For comparison, a group only activated without the injection of spermatid was used as an active control.

The embryonic cleavage rate of the embryos injected with the round spermatids had no difference from the active control, but the blastocyst formation rate and the hatching rate were higher than those of the active control, respectively. Specifically, 24 of 56 embryos formed blastocysts, and 12 of them were hatched. 50 embryos of the active control were activated, 12 of them formed blastocysts and only 3 of them were hatched, thus indicating a statistically significant difference from the embryos of this Example. The blastocyst formation rate of the embryos of this Example: 24/56 (42.9%); the hatching rate of the embryos of this Example: 12/24 (50%); the blastocyst formation rate of the active control: 12/50 (24.0%); and the hatching rate of the active control: 3/12 (25.0%); P<0.05.

### INDUSTRIAL APPLICABILITY

As described above, the present invention has found that germ-line stem cells isolated from mammalian testis were cultured in the embryonic stem cell culture medium, they will form colonies so that the isolation and proliferation of the germ-line stem cells will be effectively achieved at the same time. In addition, the present invention has found that encapsulation of the isolated germ-line stem cells with calcium alginate and co-culture with peritubular cells makes the differentiation of them effective. Thus, the methods according to the present invention can be effectively used for studies on germ-line stem cells, the treatment of male infertility, and the production of transgenic animals by the use of sperm, and the like.

1. A method for the in vitro isolation and proliferation of mammalian germ-line stem cells, the method comprising the step of culturing a cell suspension isolated from the testis of mammals in an embryonic stem cell culture medium.

2. The method of claim 1, wherein the embryonic stem cell culture medium is DMEM (Dulbecco's modified Eagle's medium; GIBCO) supplemented with fetal bovine serum, nonessential amino acid, 2-mercaptoethanol, human leukemia inhibitory factor, bFGF and forskolin.

3. The method of claim 1, wherein the embryonic stem cell culture medium is DMEM (Dulbecco's modified Eagle's medium) supplemented with 15% fetal bovine serum, 1% nonessential amino acid, 10 μM 2-mercaptoethanol, 1500 U/ml of human leukemia inhibitory factor, 4 ng/ml of bFGF and 10 μM forskolin.

4. The method of claim 1, wherein the mammals are mice or human beings.

5. The method of claim 1, wherein the cell suspension is prepared by extracting testicular tissue from the mammals and subjecting the testicular tissue to two-step enzymatic digestion process.

6. The method of claim 1, which additionally comprises the step of treating the cells cultured in the embryonic stem cell culture medium with an enzyme so as to separate the cells into single cells and subculturing the single cells.

7. The method of claim 6, wherein the subculture is performed 5-7 times.

8. The method of claim 6, wherein the enzyme is trypsin.

9. The method of claim 1, which additionally comprises the steps of mechanically sectioning the cells cultured in the embryonic stem cell culture medium and subculturing the sectioned cells in a prepared feeder layer.

10. The method of claim 9, wherein the prepared feeder layer is prepared by excluding germ-line stem cells from the intratesticular peritubular cells cultured in the embryonic stem cell culture medium.

11. A method for the in vitro differentiation of germ-line stem cells, the method comprising encapsulating the germ-line stem cells and Sertoli cells of mammals with calcium alginate and three-dimensionally culturing the encapsulated cells.

12. The method of claim 11, wherein the encapsulated cells are co-cultured with mammalian testicular cells containing peritubular cells.

13. The method of claim 11, wherein the culture is conducted in a HEPES-buffered DMEM/F12 medium supplemented with insulin-transferrin-selenium solution, vitamin C, vitamin E, retinoic acid, retinol, pyruvate, recombinant human FSH, testosterone, antibiotic-antimycotic (ABAM) (containing penicillin, streptomycin and amphotericin B) and BCS.

14. The method of claim 12, wherein the culture is conducted in a HEPES-buffered DMEM/F12 medium supplemented with insulin-transferrin-selenium solution, vitamin C, vitamin E, retinoic acid, retinol, pyruvate, recombinant human FSH, testosterone, antibiotic-antimycotic (ABAM) (containing penicillin, streptomycin and amphotericin B) and BCS.

15. The method of claim 12, wherein the mammalian testicular cells containing peritubular cells are prepared by culturing testicular cells in a DMEM/F12 medium supplemented with bovine calf serum, FSH and testosterone.

16. Isolated, undifferentiated germ-line stem cells obtainable by a method according to claim 1.

17. Isolated, differentiated germ-line stem cells isolated by a method according to claim 11.

18. A method for the treatment of male infertility, comprising administering to an individual an effective amount of germ-line stem cells according to claim 16.

19. The method of claim 18, wherein the male infertility is selected from the group consisting of oligozoospermia, asthenozoospermia, teratozoospermia and azoospermia.

20. The method of claim 19, wherein the azoospermia is non-obstructive azoospermia.

21. A composition for the treatment of male infertility, comprising germ-line stem cells according to claim 16.

22. A method for determining the presence of germ-line stem cells, the method comprising the steps of:
(a) extracting testicular tissue from mammals;
(b) subjecting the testicular tissue to a two-step enzymatic digestion process so as to prepare cell suspension; and
(c) culturing the cell suspension in a stem cell culture medium and determining colony formation.

23. A method for diagnosing the differentiation ability of germ-line stem cells, the method comprising the steps of:
(a) extracting testicular tissue from mammals;
(b) subjecting the testicular tissue to a two-step enzymatic digestion process so as to prepare cell suspension;
(c) culturing the cell suspension in a stem cell culture medium;
(d) encapsulating the cultured germ-line stem cells and Sertoli cells with calcium alginate and three-dimensionally culturing the encapsulated cells.


SEQUENCE LISTING (10 sequences)

SEQ ID NO: 1 - DNA, 20 bp, Artificial
  Primer with homology to Oct-4
accatgtttctgaagtgccc

SEQ ID NO: 2 - DNA, 20 bp, Artificial
  Primer with homology to Oct-4
gctcctgatcaacagcatca

SEQ ID NO: 3 - DNA, 24 bp, Artificial
  Primer with homology to c-kit
ctggtggttcagagttccatagac

SEQ ID NO: 4 - DNA, 24 bp, Artificial
  Primer with homology to c-kit
tcaacgaccttcccgaaggcacca

SEQ ID NO: 5 - DNA, 20 bp, Artificial
  Primer with homology to TP-1
tggcatgaggagaggcaaga

SEQ ID NO: 6 - DNA, 20 bp, Artificial
  Primer with homology to TP-1
gctcattgccgcatcacaag

SEQ ID NO: 7 - DNA, 17 bp, Artificial
  Primer with homology to 18S ribosomal protein.
agatgatcgagccgcgc

SEQ ID NO: 8 - DNA, 23 bp, Artificial
  Primer with homology to 18S Ribosomal Protein
gctaccagggcctttgagatgga

SEQ ID NO: 9 - DNA, 27 bp, Artificial
  Primer with homology to TH2B
ctctctagaaaggttacttgagccatg

SEQ ID NO: 10 - DNA, 25 bp, Artificial
  Primer with homology to TH2B
ctctatagatgccggtgtcggggtg